(19) United States
(12) Patent Application Publication
(10) Pub. No.: US 2019/0343494 A1
(43) Pub. Date: Nov. 14, 2019

(54) AUTOMATED CELL COLLECTION AND SMEARING

(71) Applicants: NORTHWESTERN UNIVERSITY, Evanston, IL (US); NORTHSHORE UNIVERSITY HEALTHSYSTEM, Evanston, IL (US)

(72) Inventors: Vadim Backman, Chicago, IL (US); Hariharan Subramanian, Chicago, IL (US); John E. Chandler, Evanston, IL (US); Maria Jose Proenca, Chicago, IL (US)

(21) Appl. No.: 16/216,091

(22) Filed: Dec. 11, 2018

Related U.S. Application Data
(63) Continuation of application No. 14/213,645, filed on Mar. 14, 2014, now abandoned.
(60) Provisional application No. 61/786,802, filed on Mar. 15, 2013.

(51) Int. Cl. A61B 10/02

(52) U.S. Cl. CPC A61B 10/02

(57) ABSTRACT

The present invention provides systems, devices, and methods for cell collection and/or cell smearing (e.g., on a slide). In particular, provided herein is an cell collection device, a translating slide holder, systems comprising such devices, and methods of use thereof.

### PRIORITY CLAIM

This present application is a continuation of U.S. patent application Ser. No. 14/213,645, filed on Mar. 14, 2014, which claims priority to U.S. Provisional Patent Application No. 61/786,802, filed on Mar. 15, 2013, entitled “AUTOMATED CELL COLLECTION AND SMEARING,” the entirety of each is incorporated by reference herein.

### TECHNICAL FIELD

The present invention provides systems, devices, and methods for cell collection and/or cell smearing (e.g., on a slide). In particular, provided herein is a cell collection device, a translating slide holder, systems comprising such devices, and methods of use thereof.

### BACKGROUND

Detection and diagnosis of a variety of diseases, such as cancer, often involves the collection and microscopic examination of a cell sample. Typically, as with the Papanicolaou (“Pap”) test, these cell samples are collected from a patient with a type of cytology brush. After collection, the cell samples must be transferred from the bristles of the brush to some medium (e.g., microscopy slide) that allows for the examination of the cells. Procedures for collecting cells from a collection site within a subject involve the cells being brushed manually by a user/operator, without a mechanism for regulating or standardizing the amount of force or pressure applied or the number of brush rotations at the sampling site. Additionally, smearing of the cells on a cytology slide is performed manually without standardized or regulated parameters. The results of these manual cell brushing and smearing operations are inconsistent, suboptimal, and/or unusable.

### SUMMARY

The present invention provides systems, devices, and methods for cell collection and/or cell smearing (e.g., on a slide). In particular, provided herein is a cell collection device, a translating slide holder, systems comprising such devices, and methods of use thereof.

In some embodiments, the present invention provides cell collection devices 100 comprising:

(a) a receiving portion 101, configured to receive a cell collection element 102; (b) a motor 201, configured to activate the cell collection element 102; and (c) a force and/or pressure sensor 202 (collectively referred to hereinafter as a “pressure sensor” whether detecting force or force per unit area) configured to detect an amount of pressure between the cell collection element 102 and a surface. The pressure sensor 202 in any embodiment described herein can detect pressure (the force applied to a unit area of surface) or force. In some embodiments, the cell collection device 100 further comprises a cell collection element 102 configured to contact a surface. In some embodiments, the cell collection device 100 further comprises a handle portion 103, configured to be gripped by a user. In some embodiments, the surface is a cell collection site. In some embodiments, the cell collection site is an anatomical surface. In other embodiments the surface is an analysis substrate 303. In some embodiments, the analysis substrate 303 is a cytology or microscopy slide. In some embodiments, the cell collection element 102 is removable and/or disposable. In some embodiments, the cell collection device 100 further comprises at least one indicator 104 that informs the user whether or not the amount of pressure detected by the pressure sensor 202 is within or outside of an optimal range. In some embodiments, the motor 201 is automatically activated when amount of pressure detected by the pressure sensor 202 is within or outside of an optimal range. In some embodiments, the motor 201 is configured to stop running if the pressure detected by the pressure sensor 202 deviates from or is outside of a pre-established or optimal range. In some embodiments, the motor 201 is configured to stop after a defined amount of time. In some embodiments, the cell collection element 102 is a cytology brush. In some embodiments, the surface is a cell collection site. In some embodiments, the cell collection site is selected from the group consisting of an organ site, a tissue, blood, saliva, sputa, spinal fluid, mucus or urine.

In some embodiments, the present invention provides translating slide holders 300 comprising: (a) a base 301; (b) a slide holder 302 atop the base 301 configured to accept an analysis substrate 303; and (c) a motor configured to translate the slide holder 302 across the base 301 when amount of pressure is within an optimal or pre-set range. In some embodiments, the translating slide holder 300 further comprises a pressure sensor 202 configured to measure the amount of pressure applied to the analysis substrate.

In some embodiments, the present invention provides a cell collection and deposition system comprising a cell collection device 100 as above and a translating slide holder 300 comprising: (a) a base 301; (b) a slide holder 302 atop the base 301 configured to accept an analysis substrate 303; and (c) a motor configured to translate the slide holder 302 across the base 301 when amount of pressure is within a pre-set or optimal range. In some embodiments, the translating slide holder 300 of the cell collection and deposition system further comprises a pressure sensor plate 401 configured to measure the amount of pressure applied to the analysis substrate.

In some embodiments, the present invention provides methods of collecting cells comprising placing a cell collection device 100 as described herein against a cell collection site, pressing the cell collection element 102 against the cell collection site until sufficient pressure to activate the motor 201 is achieved; and holding the force/pressure constant while the motor 201 activates (e.g. agitates, brushes, vibrates, oscillates, rotates, etc.) the cell collection element 102.

In some embodiments, the present invention provides methods of plating a cell sample comprising: placing an analysis substrate 303 in the slide holder 302 of a translating slide holder 300 as above, placing the cell collection element 102 containing a cell sample atop an analysis substrate, pressing the cell collection element 102 against the analysis substrate 303 until sufficient pressure to activate the motor 201 is achieved, and holding the pressure constant while the motor translates the analysis substrate 303 beneath the cell collection element 102.

### BRIEF DESCRIPTION OF THE DRAWINGS

FIG. 1 shows an exemplary embodiment of an automated cell collection device 100 designed to automate smearing and cell collection operations. The collection device 100 is capable of activating the cell collection element 102, detecting pressure applied to the cell collection element 102, and providing the user with feedback on the pressure they are applying during a cell collection or smearing procedure.

FIG. 2 shows an exploded view of an exemplary device, showing components for automated cell collection. A cell collection element 102 mounts to a motor 201 and is cantilevered at a piezoelectric collar 202 allowing forces or pressures applied to the cell collection element 102 to be measured. Indicators 104 (e.g., Pressure status LEDs) provide the user with feedback and the acquisition/deposition button 204 allows the user to switch operation modes and power the device on and off.

FIG. 3 shows a rendering of an exemplary embodiment of a translating slide holder 300, showing translation between two different slide positions. This device securely holds an analysis substrate 303 for smearing with the cell collection device 100. Pressure sensors 202 on the slide holder 300 and/or cell collection device 100 synchronize translation of the analysis substrate 303 and activation of the cell collection element 102 to create an ideal smear each time.

FIG. 4 shows a non-limiting rendering of an exemplary system comprising a motorized slide holder 302 with a pressure sensor plate 401 and the motorized cell collection device 100 with force feedback. When the optimum pressure is applied during cell collection, sensors in the cell collection device 403 trigger a motor 201 to activate the cell collection element 102, acquiring cells from the desired location. The cell collection element 102 is then applied to the analysis substrate 303 in the slide holder 302 and the pressure feedback to both devices triggers simultaneous translation of the analysis substrate 303 and activation of the cell collection device 100.

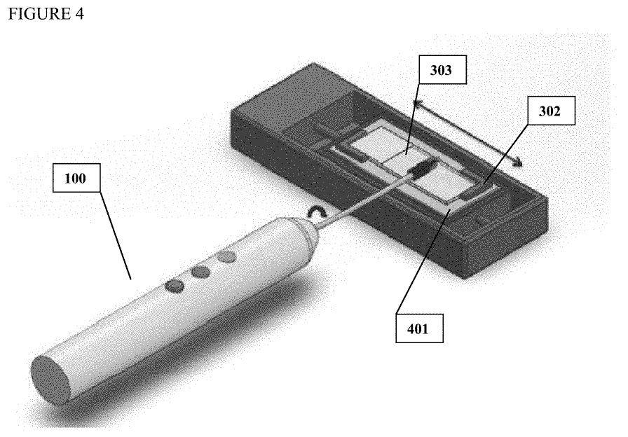

### DETAILED DESCRIPTION

In various embodiments, the present invention provides systems, devices, and methods for cell collection and/or cell smearing (e.g., on a slide). In one embodiment, provided herein is a cell collection device 100, a translating slide holder 300, systems comprising such devices, and methods of using the same. In some embodiments, the present invention addresses issues of sub-optimal and/or inconsistent cell collection with a two-device system. In some embodiments, the devices and system not only automate portions of the collection and smearing processes, but also utilize quantitative, real-time feedback to ensure optimal and consistent results. In some embodiments, the systems and devices enhance cell collection in situations in which liquid cytology is not an option or is suboptimal. In embodiments when liquid cytology is utilized, the cell collection device 100 finds use in cell collection and deposition into liquid media.

### Cell Collection Device

In some embodiments, systems, devices, and methods address inconsistencies in sample collection procedures for: i) acquiring cells with a cell collection element 102, and/or ii) smearing cells onto an analysis substrate 303 (e.g., glass microscopy slide). In some embodiments of the present invention, a motor 201 to activate the cell collection element 102 is provided. The motor can impart rotation, brushing, oscillation, agitation or vibration of the cell collection element 102. In certain embodiments, the motor 201 is useful for cell collection (e.g., from a subject) and/or cell smearing (e.g., onto a microscopy slide). In some embodiments, the cell collection device 100 comprises an activatable cell collection element 102 that is configured for collection of cells upon activation (e.g. rotation, vibration, agitation, oscillation, etc.) of the cell collection element 102 against a surface on or within a subject. In some embodiments, the cell collection device 100 comprises an activatable cell collection element 102 that is configured for collection of cells upon activation (e.g. rotation, vibration, agitation, etc.) of the cell collection element 102 against a surface outside of a subject. In some embodiments of the present invention, the cell collection element 102 is a brush, a sponge, a scraper, a swab, among others. In some embodiments, the cell collection element 102 is a cytology brush.

In other embodiments, the invention provides a cell collection device 100 that does not comprise such a cell collection element 102; rather, the cell collection device 100 is a holder/rotator of such a cell collection element 102 (e.g., removable and/or disposable cell collection element 102 and a reusable device).

In some embodiments, the cell collection device 100 further comprises a handle portion 103, configured to be gripped by a user. In other embodiments, the cell collection device 100 is attached to an instrument, such as an endoscope or a robotic apparatus. In yet other embodiments, the cell collection device 100 is attached to a wearable device, such as a glove.

In certain embodiments, a feature of the cell collection device 100 is a force feedback to measure forces at the end of the cell collection element 102 (e.g., cantilevered end). In certain embodiments, the force feedback mechanism measures, detects and/or quantifies forces or pressures applied to the end of the cell collection element 102. In some embodiments, the force feedback mechanism is a pressure sensor 202. In some embodiments, the device reports to a user (e.g., optically (e.g., LED), audibly, tactically (e.g., vibration of device)) when optimal force/pressure for collecting cells has been applied to the surface, and cell collection should commence. In some embodiments, the user activates the cell collection element 102 manually using an at least one activation/deposition button 105, upon receiving a signal from the device. In other embodiments, rotation or another motion (e.g., agitation, oscillation, vibration, etc.) automatically commences upon the cell collection device 100 sensing that the applied force/pressure is within the acceptable and/or desired range. In certain embodiments, the cell collection device 100 selects an appropriate and/or optimal activation (e.g. rotation, agitation, brush, etc.) speed and/or duration for the applied pressure detected by the cell collection device 100 (e.g., less pressure or force =longer duration or higher speed), such that cell collection is consistent despite variability in applied pressure.

In some embodiments, the cell collection device 100 provides feedback to the user when the pressure applied by the cell collection element 102 is within a desirable or pre-set range. In some embodiments, a desirable or pre-set pressure range results in collection of an optimal cell number and/or cell type (e.g., as experimentally determined). In some embodiments, the user initiates and performs the collection upon receiving a signal from the device. In other embodiments, when the applied pressure is within a desirable range, the cell collection device 100 automatically performs cell collection by activating (e.g. rotating) the cell collection element 102 at the required sampling rate.

In some embodiments, the cell collection device 100 provides feedback to the user that more pressure is needed to achieve a desirable or pre-set pressure range for the collection of an optimal cell number and/or cell type (e.g., as experimentally determined). In other embodiments, the cell collection device 100 provides feedback to the user that less pressure is needed to achieve a desirable or pre-set pressure range for the collection of an optimal cell number and/or cell type (e.g., as experimentally determined).

In some embodiments, a cell collection device 100 automates and/or regulates one or more aspects of cell acquisition (e.g., to ensure consistent brushing (or agitating, vibrating, etc.) and results). In some embodiments, an automated cell collection device 100 ensures that the brushing (or agitating, vibrating, etc.) of a cell collection site is automatic and consistent. For example, the cell collection device 100 ensures that a desirable pressure is utilized (e.g., by alerting a user when the desired pressure range is reached, by activating the cell collection device 100 when the desired pressure is achieve or deactivating the cell collection device 100 when outside of the range). The cell collection device 100 may also automate/regulate one or more of: counting the number of cell collection element 102 rotations, oscillations or vibrations (e.g., and shutting off after the appropriate/desired number, alerting a user when the desired number has been reached), setting the rotation, oscillation or vibration speed (e.g., according to the pressure applied (e.g., the exact pressure within the range), etc.

In some embodiments, a cell collection device 100 functions as a cell smearing device in addition to being a cell collection device 100. Similar to cell collection, the process of smearing cells on an analysis substrate 303 can be optimized for the ideal pressure of the cell collection element 102 against the analysis substrate 303, number of cell collection element 102 rotations, oscillations or vibrations, speed of rotation, slide translation speeds, etc. In some embodiments (e.g., because the device is useful for both collecting and smearing), the cell collection device 100 allows a user to perform cell smearing immediately following cell collection, thereby preventing drying of the sample on the cell collection element 102. In some embodiments, smearing is completely analogous to collecting, in that upon achieving the desired pressure, the cell collection device 100 is activated manually using an at least one activation/deposition button 105 (e.g., upon a user receiving a signal from the device) or automatically. In other embodiments, smearing is performed by dragging the cell collection element 102 across an analysis substrate 303 (or translating an analysis substrate 303 under a cell collection element 102), without activation of the cell collection device 100. Whether or not a cell collection device 100 is activated during smearing, a cell collection device 100 pressure sensor 202 ensures that optimal smearing technique is consistently achieved.

In some embodiments, a cell collection device 100 comprises a housing, for example to provide a structure for handling by a user, to secure the cell collection element 102, and/or to protect the inner working elements (e.g., motor, sensor(s), electronics, wires, etc.). A housing may be ergonomically shaped and sized to allow for comfortable handling by a user (e.g., to allow user to apply consistent pressure). The housing may be made of any suitable material (e.g., plastic, metal, glass, combinations thereof, etc.). In some embodiments, a cell collection device 100 comprises one or more status indicators 104 (e.g., lights (e.g., LEDs), speaker, display screen, vibrator, etc.) to indicate the pressure range of the cell collection element 102 that the user applies to the cell collection site. For example, a green light indicates that the pressure is within the acceptable range while red indicates that the pressure is outside the ideal range. In some embodiments, in order to ensure the user correctly swabs the particular cell collection site, the collection device 100 comprises an illumination LED ring that is present at the tip of the cell collection device 100. In some embodiments, a cell collection device 100 comprises one or more actuators, switches, buttons, etc. In some embodiments, a first actuators is to power ON/OFF the device, while a second actuators is to switch between operating in cell collection mode and smearing mode. In some embodiments, a motor 201 (e.g., DC motor) is provided to activate the cell collection element 102. In some embodiments, a force sensing collar (e.g., made of Piezoelectric sensor or other similar pressure sensor 202) is contained at the tip of the housing (e.g., just below the LED illumination ring). In some embodiments, cell collection element 102 is inserted through the force sensing collar in order to sense the deflection of the cantilevered cell collection element 102. In certain embodiments, forces within the threshold range triggers the motor 201 to spin or alert the user to initiate cell collection, thereby driving the cell collection element 102 to collect cells from the collection site. If the pressure is outside the required range, the motor 201 does not spin (e.g., cease to spin) or the user receives a signal that pressure is outside the range (or a lack of an affirmative signal). In some embodiments, a power source (e.g., battery pack) or power source connection (e.g., A/C adapter) is encased inside the housing to power the electronics and the motor 201.

In some embodiments, a cell collection element 102 is inserted into the cell collection device 100 through the force sensing collar. Inside the device, the handle-end of the cell collection element 102 connects to a motor 201 that is used to activate the cell collection element 102. A force sensing collar that is present at the tip of automated cell collection device 100 senses the force associated with the application of the cell collection element 102 to a cell collection site. In some embodiments, in order for the cell collection device 100 to operate (or to receive a signal to operate the device), the user selects either the collection mode or the smearing mode. In some embodiments, once the cell collection mode is selected on the cell collection device 100, the ring LEDs that are present at the tip of the cell collection device 100 illuminates to indicate whether appropriate pressure is being applied to the cell collection element 102.

In some embodiments, once the cell collection site is selected, the user uses the cell collection device 100 to introduce the cell collection element 102 into the cell collection site. Once the user applies pressure at the cell collection site, the pressure is detected by the pressure sensor 202 that is present in the cell collection device 100.

In certain embodiments, when the pressure detected by the pressure sensor 202 reaches a certain threshold ideal for collecting cells (or is within such a range), the motor 201 is activated to spin the cell collection element 102 at the cell collection site. In such embodiments, the motor 201 does not spin until the threshold pressure is met. In some embodiments, if the threshold pressure is not maintained, the motor 201 ceases to spin until the correct pressure is reapplied. In some embodiments, status LEDs or another signaling mechanism (e.g., display screen, audible signal, etc.) indicates to the user that the pressure is too low. Similarly, if the pressure that applied to the cell collection site exceeds the limit for the acceptable range, the motor 201 stops until pressure is decreased. In such a case, an indicator 104 that pressure is outside of the range is active (e.g., red status LED will be ON) indicating that the pressure applied is too high. If a pressure is within a normal range, the motor 201 spins thereby activating the cell collection element 102. In some embodiments, after a fixed number of rotations (agitations, vibrations, oscillations, brushes, etc.), the motor 201, and hence the rotation (agitations, vibrations, oscillations, brushes, etc.), stops and the status LEDs indicate that the cell collection from the cell collection site is complete.

In other embodiments, when the pressure detected by the pressure sensor 202 reaches a certain threshold ideal for collecting cells (or is within such a range), a user receives an indication that appropriate pressure is being applied. In some embodiments, the motor 201 is taken out of a locked mode when the appropriate pressure range is achieved. In such embodiments, the user is then free to activate the motor 201 and cause the cell collection element 102 to activate at the cell collection site. In some embodiments, the motor 201 does not spin until the threshold pressure is met. In other embodiments, the motor 201 can be activated at any time by the user, but the user is signaled (e.g., audibly, visibly, etc.) when the pressure is appropriate to begin collection. In some embodiments, if the threshold pressure is not maintained, the motor 201 ceases to spin until the correct pressure is reapplied. In some embodiments, if the threshold pressure is not maintained, an indicator 104 (e.g., LED, light, display, sound) alerts the user. In some embodiments, status LEDs or another signaling mechanism (e.g., display screen, audible signal, etc.) indicates to the user that the pressure is too low. Similarly, if the pressure that applied to the cell collection site exceeds the limit for the acceptable range, an indicator 104 that pressure is outside of the range is active (e.g., red status LED will be ON) indicating that the pressure applied is too high. In some embodiments, after a fixed number of rotations, an indicator 104 (e.g. LEDs, audible indicator, etc.) alerts the user that collection is complete and the motor 201 should be turned off and the cell collection element 102 removed from the site.

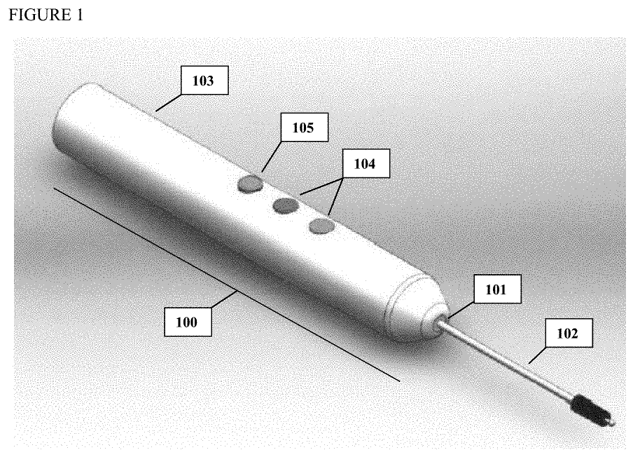

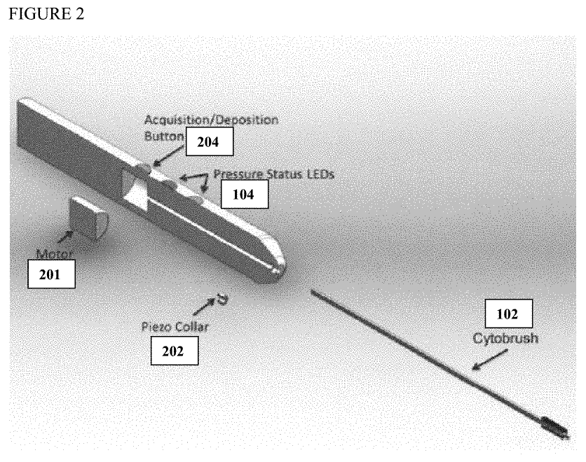

Renderings of an exemplary cell collection tool are shown in FIGS. 1 and 2. These figures are intended to provide one exemplary embodiment, and to not be limiting.

### Translating Slide Holder

In some embodiments, systems, devices, and methods address inconsistencies in sample collection procedures applying (e.g., smearing) collected cells from a cell collection element 102 onto a surface (e.g., glass microscopy slide). To address these inconsistencies, a translating slide holder 300 is provided. In some embodiments, smearing of cells from a cell collection element 102 to plate is facilitated or enhanced by the use of a ‘smart’ slide holder 300. In some embodiments, the slide holder 300 senses contact between the cell collection element 102 and the analysis substrate 303. In some embodiments, a pressure sensor plate 401 senses contact between the cell collection element 102 and the analysis substrate 303. In some embodiments, upon the appropriate pressure (e.g., pressure range) being achieved, a portion of the translating slide holder 300 shifts position (e.g., translates) causing the slide to move beneath the cell collection element 102, under the desired pressure, thereby smearing cells from the cell collection element 102 to the slide (e.g., in a consistent and effective manner). In some embodiments, a translating slide holder 300 may be used with a manual cell collection element 102 or an automated cell collection element 102. In some embodiments, in which automated translating slide holder 300 and cell collection device 100 are used together (e.g., in a system), each device senses the appropriate pressure (or range), and the cell collection element 102 is activated and the slide begins translating upon achievement of such pressure.

In some embodiments, an automated cell smearing device (e.g., translating slide holder) comprises a mechanical slide holder 300, which serves to secure an analysis substrate 303 (e.g., microscopy slide), and a translating stage to support the analysis substrate 303. In some embodiments, a linear actuator/motor is present within the device to translate both the analysis substrate 303 and the slide holder 302. In certain embodiments, a pressure sensor plate 401 is installed on top of the translating stage to measure the pressure applied to the analysis substrate 303 and to trigger the motion of the stage when the forces are within an ideal range. In some embodiments, a power source (e.g., battery pack) or power source connection is contained within the cell smearing device to power the electronics and/or actuator/motor.

In some embodiments, methods are provided for using a translating slide holder 300 to apply cells to an analysis substrate 303. In some embodiments, a clean analysis substrate 303 is loaded onto the translating stage of the translating slide holder device 300. In some embodiments in which the translating slide holder 300 is used in conjunction with an automated cell collection device 100, the automated cell collection device 100 is set to ‘smearing mode’. The translating slide holder 300 may also be used with a manual cell collection device 100. In some embodiments, immediately following cell collection, the cell collection element 102 is pressed against one end of the analysis substrate 303. In some embodiments, the ‘start end’ of the analysis substrate 303 is indicated on the analysis substrate 303 and/or holder. In some embodiments, once the cell collection element 102 is placed against the analysis substrate 303, the pressure sensor plate 401 senses the pressure on the analysis substrate 303 and triggers the analysis substrate 303 to begin translating. In some embodiments, the analysis substrate 303 does not begin translating until the appropriate amount of pressure (e.g., for a specific application) is detected. In some embodiments, a translating slide holder 300 comprises at least one indicator (e.g., LED, display, etc.) that prompts a user to apply more or less pressure.

In embodiments, in which a manual cell collection element 102 is utilized, a user either holds the cell collection element 102 steady or activates the cell collection element 102 as the analysis substrate 303 is translated beneath it. In embodiments, in which a cell collection device 100 is utilized along with a translating slide holder 300 (and rotation during smearing is desired), the pressure detected by the cell collection device 100 triggers its motor 201 to activate, resulting in the cell collection element 102 being activated while the analysis substrate 303 translates. In some embodiments, the simultaneous rotation of the cell collection device 100 and translation of the cell smearing device provides a uniform smear across the length of the analysis substrate 303.

In some embodiments, following the smearing of cells onto a slide, the smeared slide is immediately processed and/or viewed.

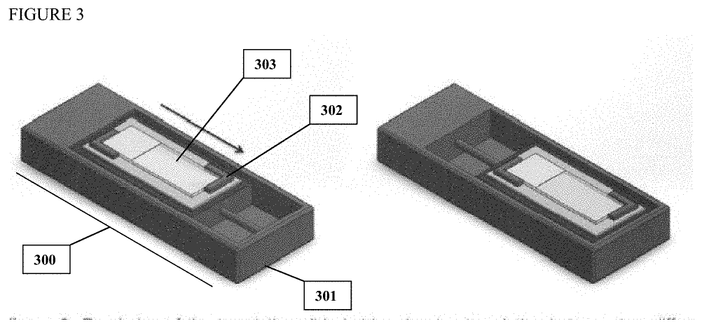

FIG. 3 provides a rendering of an exemplary translating slide holder 300. This figure is intended to provide one exemplary embodiment, and to not be limiting.

### Systems

The present invention provides an automated cell collection/smearing device (e.g., cell collection element 102 rotation device) as well as a translating slide holder 300. In some embodiments, these devices are used individually. For example, in some embodiments, a translating slide holder 300 finds use with a manual cell collection element 102 or other specimen collection tool. Likewise, in some embodiments, a cell collection device 100 finds use in collecting cells for depositing in liquid media or on a surface through another mechanism. However, in certain embodiments, systems are provided comprising both an automated cell collection/smearing device (e.g., cell collection device 100) as well as a translating slide holder 300. In some embodiments, the functionalities of the two devices are complimentary, and serve to provide consistent and optimal cell collection and deposition onto slides.

In some embodiments, when the two devices are used together, each device senses the pressure applied by the cell collection element 102 onto the analysis substrate 303. Each device is configured to begin movement (e.g., rotation, translation) upon a threshold pressure being reached. In some embodiments, both devices are configured to activate at the same pressure, such that rotation and translation begin simultaneously. In some embodiments, the translating slide holder 300 and cell collection device 100 are configured to initiate movement upon reaching different pressure thresholds or ranges. In other embodiments, one device provides feedback to the other to ensure that rotation and translation begin simultaneously.

### Applications

In some embodiments, devices and systems provided herein find use in the collection of biological samples from a subject. In some embodiments, samples comprise cells, tissues, pathogens, blood, sera, plasma, saliva, sputa, cerebrospinal, mucus (e.g., cervical), urine, combinations thereof, etc.

Although the combination of the automated cell collection/smearing device (e.g., cell collection device 100) and translating slide holder 300 can be used to prepare cytology slides, the cell collection device 100 on its own can be used for ‘liquid cytology preparations’. For these liquid cytology preparations, after the cell collection site is brushed (agitated, vibrated, etc.) using the cell collection device 100 the cell collection element 102 is submerged in the solution of choice (e.g., cytolite in case of ThinPrep) to release the cells into the solution. Once submerged, the motor 201 can dislodge the material from the cell collection element 102. The pressure sensor 202 (e.g., piezoelectric collar) is driven to vibrate the cell collection element 102 to further dislodge the cell material into the solution.

1. A cell collection device comprising: (a) a receiving portion, configured to receive a cell collection element; (b) a motor, configured to activate the cell collection element; and (c) a pressure sensor configured to detect an amount of pressure applied on a surface by the cell collection element.

2. The device of claim 1, further comprising a handle portion, configured to be gripped by a user.

3. The device of claim 1, further comprising the cell collection element configured to engage the surface.

4. The device of claim 3, wherein the cell collection element is removable and/or disposable.

5. The device of claim 1, further comprising at least one indicator that informs a user whether or not an amount of pressure detected by the pressure sensor is within or outside of a pre-established or optimal range.

6. The device of claim 1, wherein the motor is automatically activated when amount of pressure detected by the pressure sensor is within an optimal range.

7. The device of claim 1, wherein the motor is configured to stop running if the pressure detected by the pressure sensor deviates from a pre-established or optimal range.

8. The device of claim 1, wherein the motor is configured to stop after a defined amount of time.

9. The device of claim 1, wherein the cell collection element is a cytology brush.

10. The device of claim 1, wherein the surface is a cell collection site or an analysis substrate.

11. The device of claim 10, wherein the cell collection site is selected from the group consisting of an organ site, a tissue, blood, saliva, sputa, spinal fluid, mucus or urine.

12. The device of claim 10, wherein the analysis substrate is a cytology slide.

13. A translating slide holder comprising: (a) a base; (b) a slide holder atop the base configured to accept an analysis substrate; and (c) a motor configured to translate the slide holder across the base when amount of pressure is within an optimal or pre-set range.

14. The translating slide holder of claim 13, further comprising a pressure sensor configured to measure an amount of pressure applied to the analysis substrate

15. The translating slide holder of claim 13, wherein the analysis substrate is a cytology slide.

16. A system comprising a cell collection device of claim 1 and a translating slide holder comprising: (a) a base; (b) a slide holder atop the base configured to accept an analysis substrate; and (d) a motor configured to translate the slide holder across the base when amount of pressure detected by the pressure sensor is within or outside of an optimal range.

17. The system of claim 16, further comprising a pressure sensor configured to measure the amount of pressure applied to the analysis substrate.

18. A method of collecting cells from a cell collection site comprising placing the device of claim 1 against a cell collection site, pressing the cell collection element against the cell collection site until sufficient pressure to activate the motor is achieved; and maintaining sufficient pressure between the cell collection element and the cell collection site to keep the motor activated for a period sufficient to collect cells.

19. The method of claim 18, wherein the cell collection site is selected from the group consisting of an organ site, a tissue, blood, saliva, sputa, spinal fluid, mucus or urine.

20. A method of plating a cell sample comprising: placing an analysis substrate in the slide holder of the translating slide holder of claim 13, placing a cell collection element containing a cell sample atop the analysis substrate, pressing the cell collection element against the analysis substrate until sufficient pressure to activate the motor is achieved, and holding force and/or pressure constant while the motor translates the analysis substrate beneath the cell collection element.

21. The method of claim 20, wherein the analysis substrate is a cytology slide.

22. The method of claim 20, wherein the cell collection element is a manual cell collection element.

23. The method of claim 20, wherein the cell collection element is an automated cell collection element configured to activate when sufficient pressure is achieved.
